(19) United States
(12) Patent Application Publication
(10) Pub. No.: US 2009/0281389 A1
(43) Pub. Date: Nov. 12, 2009

(54) Device, system, and method for adaptive imaging

(76) Inventor: Gavriel J. Iddan, Haifa (IL)

Correspondence Address: Pearl Cohen Zedek Latzer, LLP, 1500 Broadway, 12th Floor, New York, NY, 10036, US

(21) Appl. No.: 11/794,578

(22) PCT Filed: Dec. 22, 2005
(86) PCT No.: PCT/IL05/01373

§ 371 Date: Jul. 2, 2007

Related U.S. Application Data
(60) Provisional application No. 60/639,963, filed on Dec. 30, 2004.

(51) Int. Cl. A61B 1/00

(52) U.S. Cl. 600/167

(57) ABSTRACT

Device, system and method for adaptive imaging and adaptive in-vivo imaging. For example, an in-vivo imaging device may include a lens and a modifier to modify a physical property or an optical property of said lens.

### FIELD OF THE INVENTION

The present invention relates to the field of imaging. More specifically, the present invention relates to devices, systems, and methods for in-vivo imaging.

### BACKGROUND OF THE INVENTION

Devices, systems and methods for in-vivo imaging of passages or cavities within a body are known in the art. An in-vivo imaging system may include, for example, an in-vivo imaging device for obtaining images from inside a body cavity or lumen, such as the gastrointestinal (GI) tract. The in-vivo imaging device may include, for example, an imager associated with units such as, for example, an illumination source, a controller or processor, a power source, a transmitter, and an antenna. Other types of in-vivo devices exist, such as endoscopes which may not require a transmitter, and in-vivo devices performing functions other than imaging.

Some in-vivo imaging devices may include an optical systems edgy, a lens assembly to focus light reflected from an illuminated body organ onto the imager. Some in-vivo imaging devices may include, for example, a lens formed of glass or other rigid material, such that the lens may have a constant depth of view, non-modifiable physical properties and non-modifiable optical properties.

### SUMMARY OF THE INVENTION

Various embodiments of the invention provide, for example, an in-vivo device including an optical system having adaptive or modifiable optical properties and/or physical properties, and a method and system for using the in-vivo device.

In some embodiments, an in-vivo imaging device may include, for example, an adaptive optical system having one or more units to allow setting, increasing, decreasing, alteration or modification of a depth of view of the in-vivo imaging device, or to allow focusing or modifying a focus distance of the in-vivo imaging device.

In some embodiments, the adaptive optical system may include, for example, an adaptive lens and a modifiers unit. The lens may be formed of an elastic, flexible or semi-flexible material, for example, a material able to be bent, shaped or curved when a pressure or force is applied, e.g., an elastomer, a plastic material, or other suitable materials.

In some embodiments, the modifier unit may be able to apply pressure, apply mechanical force, apply electric force, apply piezoelectric force or cause a piezoelectric effect, apply elastic force, or otherwise affect the shape, dimensions or size of the adaptive lens.

In some embodiments, for example, the modifier unit may apply pressure or force causing the adaptive lens to expand, bend, shrink, inflate, deflate, have an increased or decreased size or volume, have an increased or decreased width or dimensions, have or alter one or more curve, become convex or concave, become relatively more convex or more concave, become relatively less convex or less concave, modify curvature, or otherwise modify one or more physical properties or optical properties of the adaptive lens. In accordance with some embodiments of the invention, these modifications may result in, for example, modifications of a focus distance, a depth of view, a focal distance and/or optical properties of the adaptive lens.

Embodiments of the invention may allow various other benefits, and may be used in conjunction with various other applications.

### BRIEF DESCRIPTION OF THE DRAWINGS

The subject matter regarded as the invention is particularly pointed out and distinctly claimed in the concluding portion of the specification. The invention, however, both as to organization and method of operation, together with objects, features and advantages thereof, may best be understood by reference to the following detailed description when read with the accompanied drawings in which:

FIG. 1 is a schematic illustration of an in-vivo imaging system in accordance with an embodiment of the invention;

FIGS. 2A-2B are schematic illustrations of an in-vivo imaging device in accordance with an embodiment of the invention;

FIG. 3 is a schematic illustration of an in-vivo imaging device in accordance with another embodiment of the invention;

FIG. 4 is a schematic illustration of an in-vivo imaging device in accordance with another embodiment of the invention; and

FIG. 5 is a flow-chart diagram of a method in accordance with an embodiment of the invention.

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

### DETAILED DESCRIPTION OF THE INVENTION

In the following description, various aspects of the invention will be described. For purposes of explanation, specific configurations and details are set forth in order to provide a thorough understanding of the invention. However, it will also be apparent to one skilled in the art that the invention may be practiced without the specific details presented herein. Furthermore, well-known features may be omitted or simplified in order not to obscure the invention.

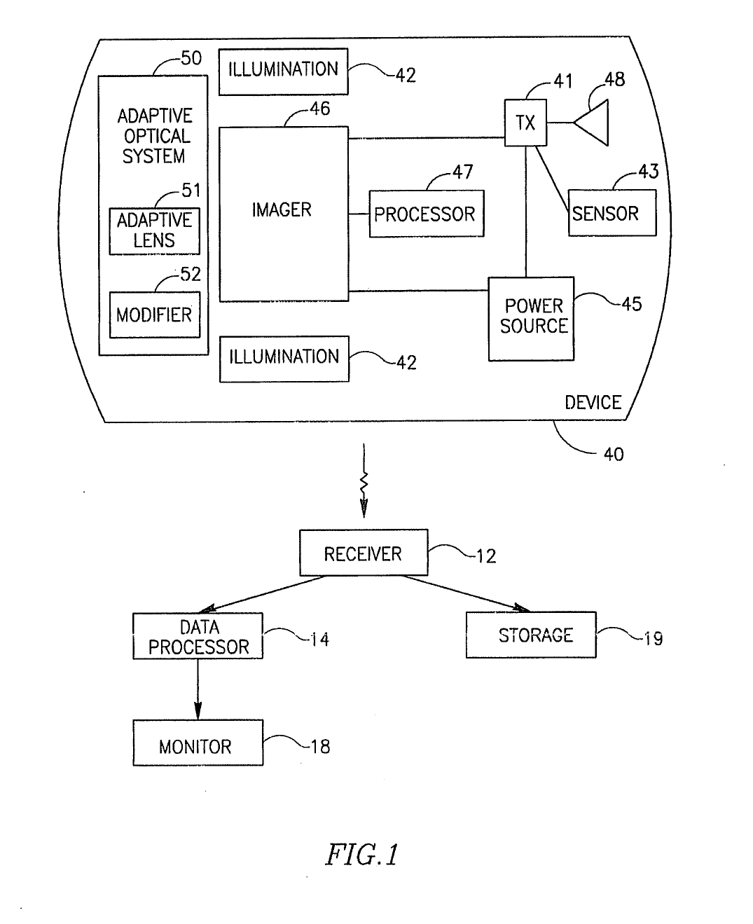

While a portion of the discussion may relate, for exemplary purposes, to setting, alteration or modification of a depth of view of an in-vivo imaging device, or to focusing or modifying a focus distance of an in-vivo imaging device, the present invention is not limited in this regard, and may be used not necessarily in the context of in-vivo imaging. Some embodiments of the present invention are directed to a typically swallowable in-vivo imaging device. Devices according to embodiments of the present invention may be similar to embodiments described in U.S. patent application Ser. No. 09/800,470, entitled “Device and System for In-vivo Imaging”, filed on 8 Mar., 2001, published on Nov. 1, 2001 as United States Patent Application Publication Number 2001/0035902, and/or in U.S. Pat. No. 5,604,531 to Iddan et al., entitled “In-Vivo Video Camera System”, and/or in U.S. patent application Ser. No. 10/046,541, filed on Jan. 16, 2002, published on Aug. 15, 2002 as United States Patent Application Publication Number 2002/0109774, all of which are hereby incorporated by reference. The device, system and method of the present invention may be utilized in conjunction with other suitable imaging devices, systems and methods for acquiring in-vivo images of a body lumen or cavity. Devices and systems as described herein may have other configurations and/or other sets of components. For example, some embodiments of the present invention may be practiced using an endoscope, needle, stent, catheter, etc. For example, an adaptive optical system according to an embodiment of the invention may be utilized in probes used for in vivo imaging, such as endoscopes. FIG. 1 shows a schematic diagram of an in-vivo imaging system in accordance with an embodiment of the present invention. In one embodiment, the system may include a device 40 having an imager 46, an illumination source 42, a power source 45, and a transmitter 41. In some embodiments, device 40 may be implemented using a swallowable capsule, or may be inserted into a patient's body by swallowing, but other sorts of devices or implementations or methods of insertion may be used. Outside a patient's body may be, for example, an image receiver 12 (including, for example, an antenna or an antenna array), a storage unit 19, a data processor 14, and a monitor 18. Transmitter 41 may operate using radio waves; but in some embodiments, such as those where device 40 is or is included within an endoscope, transmitter 41 may transmit data via, for example, wire, optical fiber and/or other suitable methods.

Device 40 typically may be or may include an autonomous swallowable capsule, but device 40 may have other shapes and need not be swallowable or autonomous. Embodiments of device 40 are typically autonomous, and are typically self-contained. For example, device 40 may be a capsule or other unit where all the components are substantially contained within a container or shell, and where device 40 does not require any wires or cables to, for example, receive power or transmit information.

In some embodiments, device 40 may communicate with an external receiving and display system (e.g., through receive, 2) to provide display of data, control, or other functions. For example, power may be provided to device 40 using an internal battery, an internal power source, or a wireless system to receive power. Other embodiments may have other configurations and capabilities. For example, components may be distributed over multiple sites or units, and control information may be received from an external source.

In one embodiment, device 40 may include an in-vivo video camera, for example, an image sensor or imager 46, which may capture and transmit images of, for example, the GI tract while device 40 passes through the GI lumen. Other lumens and/or body cavities may be imaged and/or sensed by device 40. In some embodiments, imager 46 may include, for example, a Charge Coupled Device (CCD) camera or imager, a Complementary Metal Oxide Semiconductor (CMOS) camera or imager, a digital camera, a stills camera, a video camera, or other suitable imagers, cameras, or image acquisition components.

In one embodiment, imager 46 in device 40 may be operationally connected to transmitter 41. Transmitter 41 may transmit images to, for example, image receiver 12, which may send the data to data processor 14 and/or to storage unit 19. Transmitter 41 may also include control capability, although control capability may be included in a separate component. Transmitter 41 may include any suitable transmitter able to transmit image data, other sensed data, and/or other data (e.g., control data) to a receiving device. For example, transmitter 41 may include an ultra low power Radio Frequency (RF) high bandwidth transmitter, possibly provided in Chip Scale Package (CSP). Transmitter 41 may transmit via antenna 48. Transmitter 41 and/or another unit in device 40, e.g., a controller or processor 47, may include control capability, for example, one or more control modules, processing module, circuitry and/or functionality for controlling device 40, for controlling the operational mode or settings of device 40, and/or for performing control operations or processing operations within device 40. In some embodiments, transmitter 41 and/or processor 47 may, for example, control or modify one or more physical properties or optical properties of an optical system or optical element of device 40, e.g., of an adaptive optical system 50 described herein.

Power source 45 may include one or mole batteries or power cells. For example, power source 45 may include silver oxide batteries, lithium batteries, other suitable electrochemical cells having a high energy density, or the like. Other suitable power sources may be used. For example, power source 45 may receive power or energy from an external power source (e.g., a power transmitter), which may be used to transmit power or energy to device 40.

Optionally, in one embodiment, transmitter 41 may include a processing unit or processor or controller, for example, to process signals and/or data generated by imager 46. In another embodiment, the processing unit may be implemented using a separate component within device 40, e.g., controller or processor 47, or may be implemented as an integral part of imager 46, transmitter 41, or another component, or may not be needed. The optional processing unit may include, for example, a Central Processing Unit (CPU), a Digital Signal Processor (DSP), a microprocessor, a controller, a chip, a microchip, a controller, circuitry, an Integrated Circuit (IC), an Application-Specific Integrated Circuit (ASIC), or any other suitable multi-purpose or specific processor, controller, circuitry or circuit. In one embodiment, for example, the processing unit or controller may be embedded in or integrated with transmitter 41, and may be implemented, for example, using an ASIC.

In some embodiments, device 40 may include one or more illumination sources 42, for example one or more Light Emitting Diodes (LEDs), “white LEDs”, or other suitable light sources. Illumination sources 42 may, for example, illuminate a body lumen or cavity being imaged and/or sensed, Data processor 14 may analyze the data received via receiver 12 from device 40, and may be in communication with storage unit 19, e.g., transferring frame data to and from storage unit 19. Data processor 14 may also provide the analyzed data to monitor 18, where a user (e.g., a physician) may view or otherwise use the data. In one embodiment, data processor 14 may be configured for real time processing and/or for post processing to be performed and/or viewed at a later time. In the case that control capability (e.g., delay, timing, etc) is external to device 40, a suitable external device (such as, for example, data processor 14 or image receiver 12) may transmit one or more control signals to device 40.

Monitor 18 may include, for example, one or more screens, monitors, or suitable display units. Monitor 18, for example, may display one or more images or a stream of images captured and/or transmitted by device 40, e.g., images of the GI tract or of other imaged body lumen or cavity. Additionally or alternatively, monitor 18 may display, for example, control data, location or position data (e.g., data describing or indicating the location or the relative location of device 40), orientation data, and various other suitable data. In one embodiment, for example, both an image and its position or location may be presented using monitor 18 and/or may be stored using storage unit 19. Other systems and methods of storing and/or displaying collected image data and/or other data may be used.

In some embodiments, in addition to or instead of revealing pathological or other conditions of the GI tract, the system may provide information about the location of these conditions. Suitable tracking devices and methods are described in embodiments of the above-mentioned U.S. Pat. No. 5,604,531 and/or United States Patent Application Publication Number US-2002-0173718, filed on May 20, 2002, titled “Array System and Method for Locating an In-Vivo Signal Source”, assigned to the common assignee of the present invention, and fully incorporated herein by reference. Other suitable location identification systems and methods may be used in accordance with embodiments of the present invention.

Typically, device 40 may transmit image information in discrete portions. Each portion may typically correspond to an image or a frame; other suitable transmission methods may be used. For example, in some embodiments, device 40 may capture and/or acquire an image once every half second, and may transmit the image data to receiver 12. Other constant and/or variable capture rates and/or transmission rates may be used.

Typically, the image data recorded and transmitted may include digital color image data; in alternate embodiments, other image formats (e.g., black and white image data) may be used. In one embodiment, each frame of image data may include 256 rows, each row may include 256 pixels, and each pixel may include data for color and brightness according to known methods. For example, in each pixel, color may be represented by a mosaic of four sub-pixels, each sub-pixel corresponding to primaries such as red, green, or blue (where one primary, e.g., green, may be represented twice). The brightness of the overall pixel may be recorded by, for example, a one byte (e.g., 0-255) brightness value in one embodiment, for example, image data may be represented using an array of 64 by 64 pixels or super-pixels or boxes, each including data indicating values for red, green (repeated twice) and blue. Other suitable data formats may be used, and other suitable numbers or types of rows, columns, arrays, pixels, sub-pixels, boxes, super-pixels and/or colors may be used.

Optionally, device 40 may include one or more sensors 43, instead of or in addition to a sensor such as imager 46. Sensor 43 may, for example, sense, detect, determine and/or measure one or more values of properties or characteristics of the surrounding of device 40. For example, sensor 43 may include a pH sensor, a temperature sensor, an electrical conductivity sensor, a pressure sensor, or any other known suitable in-vivo sensor.

In accordance with some embodiments of the present invention, device 40 may include adaptive optical system 50 having one or more optical elements, for example, one or more lenses or composite lens assemblies, one or more suitable optical filters, or other suitable optical elements. Optical system 50 may, for example, focus or aid in focusing light reflected onto imager 46, and/or may perform other light processing operations Adaptive optical system 50 may include, or may be operatively associated with, a mechanism to modify a physical property or optical property of adaptive optical system, as described herein.

In some embodiments, adaptive optical system 50 may include one or more units or sub-units allowing setting, increasing, decreasing, alteration or modification of a depth of view or other physical properties or optical properties of in-vivo imaging device 40 and/or imager 46, or to allow focusing or modifying a focus distance of in-vivo imaging device 40 and/or imager 46, as described herein. For example, in some embodiments, adaptive optical 50 may allow an extended depth of view, an adaptive depth of view, a variable depth of view, a modifiable of changeable depth of view, an extended Focus distance or focal distance, an adaptive focus distance or focal distance, a variable focus distance or focal distance, or a modifiable of changeable focus distance or focal distance. In some embodiments, adaptive optical system 50 may include, for example, one or more lenses 51 operatively connected to one or more modifiers 52. Lens 51 may be formed of an elastic, flexible or semi-flexible material, for example, a material able to be bent, shaped or curved when a pressure is applied, e.g., an elastomer, a plastic material, or other suitable materials.

Modifier 52 may include one or more units able to apply pressure, apply mechanical force, apply electric force, apply piezoelectric force or cause a piezoelectric effect, apply elastic force, or otherwise affect the shape or size of lens 51. For example, modifier 52 may apply pressure or force causing lens 51 to expand, bend, shrink, inflate, deflate, have an increased or decreased size or volume, have an increased or decreased width or dimensions, have one or more curve, become convex or concave, become relatively more convex or more concave, become relatively less convex or less concave, or otherwise modify one or more physical properties or optical properties of lens 51. These modifications may result in, for example, modifications of a focus distance, a depth of view, a focal distance and/or optical properties of lens 51.

In some embodiments, modifier 52 may set or modify one or more optical properties of lens 51 in response to a pre-defined command or signal, in response to a triggering event, or when pre-defined criteria or conditions are met. This may include, for example, detection of a pre-defined substance (e.g., blood) by device 40, determination of a condition (e.g., temperature, pressure or pH) by sensor 43 of device 40, or other conditions. In some embodiments, sensor 43, imager 46, transmitter 41 or processor 47 may send a signal indicating that the pre-defined criteria or conditions are met, and that one or more optical properties of physical properties of adaptive lens 51 may be modified. In one embodiment, the modification may be performed in substantially real time and while device 40 is in-vivo, for example, in response to an external command or signal transmitted to device 40, or in accordance with a pre-defined focusing algorithm, focusing scheme, or auto-focusing algorithm.

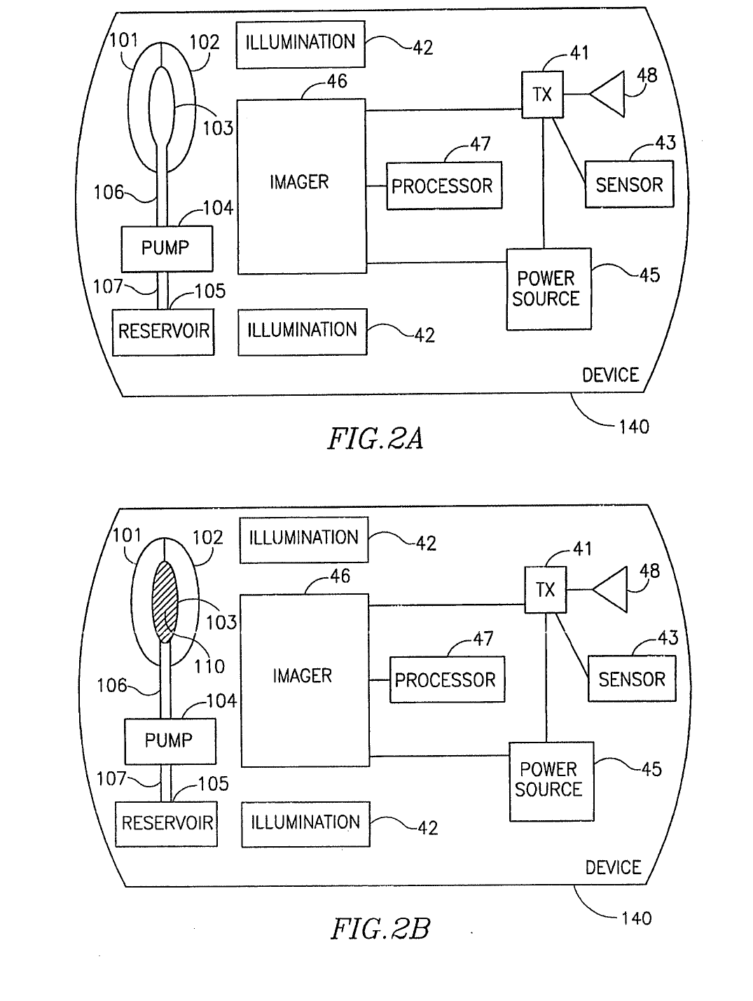

FIGS. 2A and 2B show schematic diagrams of an in-vivo imaging device 140 in accordance with an embodiment of the present invention. Device 140 may be an example of device 40 of FIG. 1. Device 140 may be used, for example, in conjunction with the in-vivo imaging system of FIG. 1.

Device 140 may include, for example, imager 46, one or more illumination sources 42, processor 47, antenna 48, power source 45, and optionally processor 47 and sensor 43. Device 140 may further include an adaptive lens 100, which may be an example of the adaptive optical system 50 of FIG. 1.

In some embodiments, adaptive lens 100 may include, for example, one or more optical elements substantially encapsulating a cavity, a chamber or a container 103. For example, adaptive lens 100 may include a portion 101 and a portion 102 which may enclose or surround container 103. Portion 101 and/or 102 may include, for example, a lens, a convex lens, a concave lens, a lens having one or more convex surfaces, a lens having one or mole concave surfaces, a lens having one or more plane surfaces, or the like. Although two portions 101 and 102 are shown, adaptive lens 100 may include other number of portions, in one embodiment, adaptive lens 100 may be formed of a single portion having an integrated container 103. Adaptive lens 100 may be formed of an elastic, flexible or semi-flexible material, for example, a material able to be bent, shaped or curved when a pressure is applied, e.g., an elastomer, a plastic material, or other suitable materials.

Container 103 may include one or more chambers or cavities enclosed within lens 100 and able to store a substance or liquid 110. In one embodiment, container 103 may be formed as an integrated part of adaptive lens 100, for example, by manufacturing an adaptive lens 100 having a cavity therein, or by forming and attaching portion 101 to portion 102 such that a cavity is enclosed within lens 100.

Liquid 110 may include, for example, an inert liquid having a relatively low mobility. Liquid 110 may be stored in a reservoir 105, and may be transferred into and/or out of container 103, for example, using a pump 104 and pipes 106 and 107. In some embodiments, pump 104 may be operatively connected to power source 45, which may provide power for the operation of pump 104, e.g., for pumping or transferring liquid 103 into and/or out of container 103. In some embodiments, pump 104 may be operatively connected to processor 47 or to other components of device 140, which may control or trigger the operation of pump 104.

In accordance with some embodiments of the invention, insertion of liquid 110 into container 103, or removal of liquid 110 from container 103, may modify the shape, size, focus or focal distance, depth of view, and/or optical properties or other physical properties of adaptive lens 100. For example, as shown in FIG. 2A, container 103 may be substantially empty of liquid 103, such that lens 100 may have a first shape and size and a first focus distance or focal distance or depth of view. Pump 104 may transfer some or all of liquid 103 from reservoir 105 into container 103, and liquid 110 may apply pressure from within container 103 outwards, such that portions 101 and 102 may bend or change their shape, as shown in FIG. 2B. As a result of the insertion of liquid 110 into container 103, adaptive lens 100 may have a second, different shape and size, and a second, different, focus distance or focal distance or depth of view. Removal of some or all of liquid 110 from container 103 by pump 104, may result in further modification of the shape and size of lens 100 and its focus distance or focal distance or depth of view.

In some embodiments, in addition to or instead of transferring liquid 110 from reservoir 105 to container 103 and vice versa, pump 104 may provide or apply additional pressure to aid in the modification of the shape and size of lens 100. For example, when substantially all of liquid 110 is transferred into container 103, pump 104 may optionally apply additional pressure on liquid 110 to allow further bending of portions 101 and/or 102.

In some embodiments, pump 104 may be used to modify one or more optical properties of adaptive lens 100 in response to a pre-defined command or signal, in response to a triggering event, or when pre-defined criteria or conditions are met. This may include, for example, detection of a pre-defined substance (e.g., blood) by device 40, determination of a condition (e.g., temperature, pressure or pH) by sensor 43 of device 40, or other conditions. In some embodiments, sensor 43, imager 46, transmitter 41 or processor 47 may send a signal to pump 104, indicating that the pre-defined criteria or conditions are met, and that one or more optical properties of physical properties of adaptive lens 100 may be modified. In one embodiment, the modification may be performed in substantially real time and while device 140 is in-vivo, for example, in response to an external command or signal transmitted to device 140, or in accordance with a pre-defined focusing algorithm, focusing scheme, or auto-focusing algorithm.

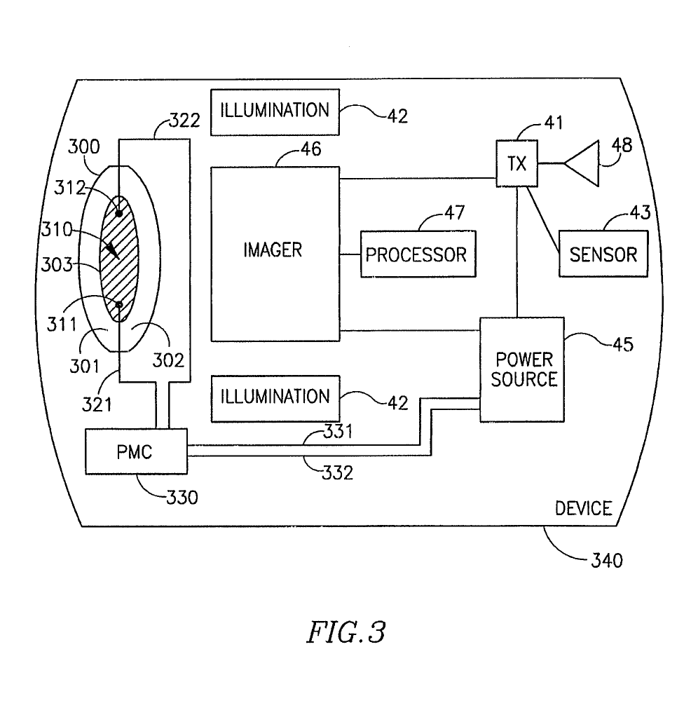

FIG. 3 shows a schematic diagram of an in-vivo imaging device 340 in accordance with another embodiment of the present invention. Device 340 may be an example of device 40 of FIG. 1. Device 340 may be used, for example, in conjunction with the in-vivo imaging system of FIG. 1.

Device 340 may include, for example, imager 46, one or more illumination sources 42, processor 47, antenna 48, power source 45, and optionally processor 47 and sensor 43. Device 340 may further include an adaptive lens 300, which may be an example of the adaptive optical system 50 of FIG. 1.

In some embodiments, adaptive lens 300 may include, for example, one or more optical elements substantially encapsulating a cavity, a chamber or a container 303. For example, adaptive lens 300 may include a portion 301 and a portion 302 which may enclose or surround container 303. Portion 301 and/or 302 may include, for example, a lens, a convex lens, a concave lens, a lens having one or more convex surfaces, a lens having one or more concave surfaces, a lens having one or more plane surfaces, or the like. Although two portions 301 and 302 are shown, adaptive lens 300 may include other number of portions; in one embodiment, adaptive lens 300 may be formed of a single portion having an integrated container 303. Adaptive lens 300 may be formed of an elastic, flexible or semi-flexible material, for example, a material able to be bent, shaped or curved when a pressure is applied, e.g., an elastomer, a plastic material, or other suitable materials.

Container 303 may include one or more chambers or cavities enclosed within lens 300 and able to store a substance or liquid 310. In one embodiment, container 303 may be formed as an integrated part of adaptive lens 300, for example, by manufacturing an adaptive lens 300 having a cavity therein, or by forming and attaching portion 301 to portion 302 such that a cavity is enclosed within lens 300.

Liquid 310 may include, for example, a liquid able to change its volume in response to a change of its temperature. For example, liquid 310 may include a liquid which volume increases when liquid 310 is heated, when heat is applied to liquid 310, or when the temperature of liquid 310 is otherwise increased. In some embodiments, liquid 310 may be substantially transparent to one or more frequencies of light or colors. In some embodiments, liquid 310 may be pre-selected to match one or more optical properties of lens 310, e.g., liquid 310 may have an index of refraction which may be substantially identical or similar to the index of refraction of lens 310.

Container 303 may further include electrodes 311 and 312 which may be in contact with liquid 310. Electrodes 311 and 312 may be part of an electric circuit able to provide electric power or current to liquid 31, e.g., to increase the temperature of liquid 310. For example, in one embodiment, electrodes 311 and 3172 may be connected through wires or connections 321 and 322, respectively, to an optional Power Management Controller (PMC) 330, which may receive power from power source 45 through wires or connection 331 and 332. In another embodiment, electrodes 311 and 312 may be otherwise connected to power source 45 or to another, dedicated power source, optionally using processor 47 or other component of device 340, and without using the optional PMC 330. In yet another embodiment, PMC 330 may be operatively connected to processor 47 or to other components of device 340, which may control or trigger the operation of PMC 3.30.

In accordance with some embodiments of the invention, PMC 330 may control, vary, turn on, turn off, increase, decrease, or otherwise regulate the power, current or voltage provided to electrodes 311 and 312. For example, PMC 330 may provide a pre-defined voltage or current to electrodes 311 and 312, e.g., a substantially constant voltage or current, or a variable voltage or current. As a result, electrodes 311 and 312 may heat up, resulting in an increase in the temperature of liquid 310, and an increase in the volume of liquid 310. As its volume increases, liquid 310 may apply pressure towards container 303 and/or lens 300, and may cause lens 300 to bend, expand, swell or change its shape or size, thereby modifying the shape, size, focus distance, focal distance, depth of view, and/or optical properties of adaptive lens 300.

In some embodiments, may disconnect an electrical circuit of which electrodes 311 and 312 are part, thereby stopping further heating of electrodes 311 and 312 and of liquid 310. This may substantially stop further bending of expansion of lens 300. Furthermore, as time elapses and electric current is not provided to electrodes 311 and 312, the temperature of liquid 310 may decrease, thereby decreasing the volume of liquid 310, reducing the pressure applied by liquid 310 on lens 300, and causing lens 300 to deflate or shrink and modify its focus distance, focal distance, depth of view, and/or optical properties.

In some embodiments, PMC 330 and/or electrodes 311 and 312 may be used to modify one or more optical properties or physical properties of adaptive lens 300 in response to a pre-defined command or signal, in response to a triggering event, or when pre-defined criteria or conditions are met. This may include, for example, detection of a pre-defined substance (e.g., blood) by device 40, determination of a condition (e.g., temperature, pressure or pH) by sensor 43 of device 40, or other conditions. In some embodiments, sensor 43, imager 46, transmitter 41 or processor 47 may send a signal to PMC 330, indicating that the pre-defined criteria or conditions are met, and that one or more optical properties of physical properties of adaptive lens 300 may be modified. In one embodiment, the modification may be performed in substantially real time and while device 340 is in-vivo, for example, in response to an external command or signal transmitted to device 340, or in accordance with a pre-defined focusing algorithm, focusing scheme, or auto-focusing algorithm.

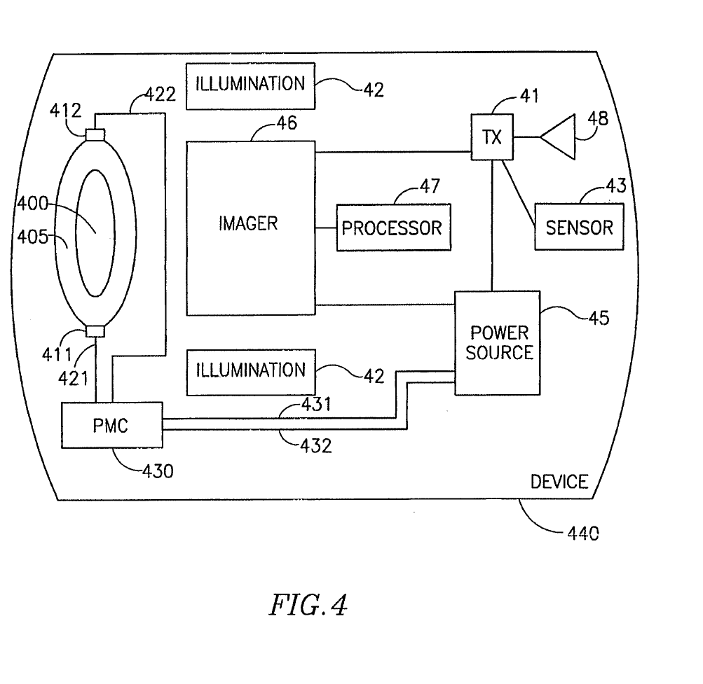

FIG. 4 shows a schematic diagram of an in-vivo imaging device 440 in accordance with yet another embodiment of the present invention. Device 440 may be an example of device 40 of FIG. 1. Device 440 may be used, for example, in conjunction with the in-vivo imaging system of FIG. 1.

Device 440 may include, for example, imager 46, one or more illumination sources 42, processor 47, antenna 48, power source 45, and optionally processor 47 and sensor 43. Device 440 may further include an adaptive lens 400, which may be an example of the adaptive optical system 50 of FIG. 1.

In some embodiments, adaptive lens 400 may include, for example, one or more optical elements. For example, adaptive lens 400 may include a lens, a convex lens, a concave lens, a lens having one or, more convex surfaces, a lens having one or more concave surfaces, a lens having one or more plane surfaces, or the like. Although a single adaptive lens 400 is shown, adaptive lens 400 may include other number of portions or elements. Adaptive lens 400 may be formed of an elastic, flexible or semi-flexible material, for example, a material able to be bent, shaped or curved when a pressure is applied, e.g., an elastomer, a plastic material, or other suitable materials.

Adaptive lens 400 may be enclosed in, or substantially surrounded by, a frame or ring 405. Although a substantially oval ring 405 is shown, ring 405 may be of other shapes; for example, ring 405 may be circular, rectangular, square. In some embodiments, ring 405 need not be closed, and may include a portion of a ring or a portion of a frame, e.g., an arc-shaped frame surrounding three-quarters or two-thirds of lens 400. In some embodiments, ring 405 may include a plurality of rings of frames. Ring 405 may be formed of, for example, a piezoelectric material, or a material which may mechanically deform or modify it shape when an electric field is applied to the material. In some embodiments, ring 405 may be firmly bonded, glued, connected, or otherwise attached to adaptive lens 400, such that when a pressure, a force or a power causes ring 405 to bend, deform or change its shape or sizes, as a result the adaptive lens 400 attached to ring 405 may modify its size or shape.

Ring 405 may be connected to, or in contact with, connectors 411 and 412. Connectors 411 and 412 may be part of an electric circuit able to provide electric power or current to ring 405, e.g., to bend or modify the shape of ring 405. For example, in one embodiment, connectors 411 and 412 may be connected through wires or connections 421 and 422, respectively, to an optional Power Management Controller (PMC) 430, which may receive power from power source 45 through wires or connection 431 and 432. In another embodiment, connectors 411 and 412 may be otherwise connected to power source 45 or to another, dedicated power source, optionally using processor 47 or other component of device 440, and without using the optional PMC 430. In yet another embodiment, PMC 430 may be operatively connected to processor 47 or to other components of device 440, which may control or trigger the operation of PMC 430.

In accordance with some embodiments of the invention, PMC 430 may control, vary, turn on, turn off, or otherwise regulate the power provided to connectors 411 and 412 and, through them, to ring 405. For example, PMC 430 may provide a pre-defined voltage or current to connectors 411 and 412 and, through them, to ring 405. In response to the electric current, e.g., due to a piezoelectric effect, ring 405 may bend, expand swell or change its shape or size. In some embodiments, adaptive lens 400 may be connected to and enclosed within ring 405, such that modification of the size or shape of ring 405 may result in modification of the size or shape of adaptive lens 400, thereby modifying the, focus distance, focal distance, depth of view, and/or optical properties of adaptive lens 400.

In some embodiments, PMC 430 may disconnect an electrical circuit of which connectors 411 and 412 are part, thereby stopping further modification of the shape or size of ring 405 and adaptive lens 400. Furthermore, as time elapses and electric current is not provided to ring 405, lens 400 may apply elastic pressure onto ring 405, thereby forcing ring 405 to modify its shape or size, e.g., to substantially the shape or size of ring 405 before the electric current was applied, or to another shape and size. This may cause further modification of the size or shape of adaptive lens 400, thereby modifying its focus distance, focal distance, depth of view, and/or optical properties.

In some embodiments, PMC 430 and/or connectors 411 and 412 may be used to modify one or more optical properties of adaptive lens 400 in response to a pre-defined command or signal, in response to a triggering event, or when pre-defined criteria or conditions are met. This may include, for example, detection of a pre-defined substance (e.g., blood) by device 40, determination of a condition (e.g., temperature, pressure or pH) by sensor 43 of device 40, or other conditions. In some embodiments, sensor 43, imager 46, transmitter 41 or processor 47 may send a signal to PMC 430, indicating that the pre-defined criteria or conditions are met, and that one or more optical properties of physical properties of adaptive lens 400 may be modified. In one embodiment, the modification may be performed in substantially real time and while device 440 is in-vivo, for example, in response to an external command or signal transmitted to device 440, or in accordance with a pre-defined focusing algorithm, focusing scheme, or auto-focusing algorithm.

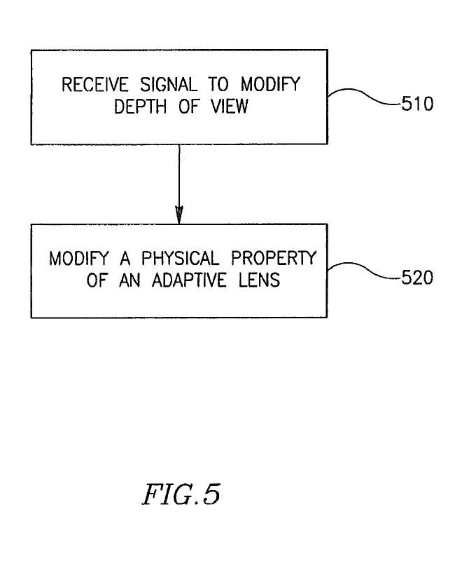

FIG. 5 is a flow-chart diagram of a method of modifying a depth of view in accordance with an embodiment of the present invention. The method of FIG. 5, as well as other suitable methods in accordance with embodiments of the invention, may be used, for example, in association with the system of FIG. 1, with device 40 of FIG. 1, with device 140 of FIGS. 2A and 2B, with device 340 of FIG. 3, with device 440 of FIG. 4, with one or more in-vivo imaging devices (which may be, but need not be, similar to device 40), and/or with other suitable devices and systems for in-vivo imaging. A method according to some embodiments of the invention need not be used in an in-vivo context.

Some embodiments of a method may, as indicated at box 510, optionally include receiving a signal, a request or a command to modify a depth of view. For example, in one embodiment, device 40 may receive an external command or signal to increase or decrease the focus distance of imager 46. In another embodiment, modifier 52 may receive an internal command or request to modify the focus distance or another optical property of lens 51, e.g., in response to a triggering event or when one or more pre-defined conditions are met.

In some embodiments, as indicated at box 520, the method may include modifying a physical property and/or an optical property of an optical element of the in-vivo imaging device, e.g., a shape, size, focal distance, focal length, focusing properties, depth of view, curvature, or the like. This may include, for example, expanding, bending, shaping, re-shaping, inflating, shrinking, applying pressure to, or otherwise modifying an adaptive lens. In one embodiment, for example, this may be performed by causing an internal pressure within the adaptive lens, e.g., using a pressured liquid or a heated liquid. In another embodiment, this may be performed, for example, by applying an external pressure on the adaptive lens, e.g., using a piezoelectric force. The operations of box 520 may result in, for example, a change in the depth of view or other optical properties of the adaptive lens.

It is noted that some or all of the above-mentioned operations may be performed substantially in real time, e.g., during the operation of the in-vivo imaging device, during the time in which the in-vivo imaging device operates and/or captures images, and/or without interruption to the operation of the in-vivo imaging device.

Other operations or sets of operations may be used in accordance with embodiments of the invention.

A device, system and method in accordance with some embodiments of the invention may be used, for example, in conjunction with a device which may be inserted into a human body or swallowed by a person. However, the scope of the present invention is not limited in this regard; for example, some embodiments of the invention may be used in conjunction with a device which may be inserted into, or swallowed by, a non-human body or an animal body.

While certain features of the invention have been illustrated and described herein, many modifications, substitutions, changes, and equivalents may occur to those of ordinary skill in the art. It is, therefore, to be understood that the appended claims are intended to cover all such modifications and changes as fall within the true spirit of the invention.

1-28. (canceled)

29. An in-vivo imaging device comprising:
a lens;
a modifier to modify an optical property of said lens; and
a controller to control the modifier.

30. The device of claim 29, wherein said modifier comprises a pump to transfer a liquid into a cavity within said lens.

31. The device of claim 29, wherein said modifier comprises a pump to apply pressure on a liquid stored within said lens.

32. The device of claim 29, wherein said modifier comprises a piezoelectric frame, a physical property of which is modifiable in response to an electric current.

33. The device of claim 29, wherein said modifier is to cause said lens to expand in response to an internal pressure.

34. The device of claim 29, wherein said modifier is to cause said lens to bend in response to an external pressure.

35. The device of claim 29, wherein said lens includes an elastic material.

36. The device of claim 29, wherein said lens includes a flexible material.

37. The device of claim 29, wherein said lens includes an elastomer.

38. The device of claim 29, wherein said adaptive lens includes plastic.

39. The device of claim 29, wherein said adaptive lens includes an expandable liquid.

40. The device of claim 29, wherein said modifier comprises an electric circuit to increase a temperature of a liquid stored within said adaptive lens.

41. The device of claim 29, wherein said device is a swallowable capsule.

42. The device of claim 29, wherein said device is an autonomous in-vivo device.

43. The device of claim 29, comprising an imager.

44. The device of claim 29, wherein said physical property comprises a curvature.

45. The device of claim 29 wherein the controller is configured to control the operation of the modifier in response to a pre-defined signal.

46. The device of claim 29 wherein the controller is configured to control the operation of the modifier in response to a triggering event said triggering event occurring externally to the device.

47. A method for in vivo imaging, the method comprising:
receiving a signal by a controller in a swallowable capsule; and
controlling an optical property of a lens in the swallowable capsule according to the received signal.

48. The method of claim 47, comprising transferring a liquid into a cavity within said lens.

49. The method of claim 47, comprising applying pressure to a liquid within said lens.

50. The method according to claim 47 comprising:
sensing a condition within a body lumen;
determining whether a pre-defined condition is met; and
based on the determination generating the signal.
